SEQUENCE LISTING (5 sequences)

SEQ ID NO: 1 - DNA, 19 bp, Artificial Sequence
  Rela shRNA
gcatgcgattccgctataa

SEQ ID NO: 2 - DNA, 19 bp, Artificial Sequence
  control shRNA
acagccacaacgtctatat

SEQ ID NO: 3 - DNA, 19 bp, Artificial Sequence
  c-Jun shRNA
agtcatgaaccacgttaac

SEQ ID NO: 4 - DNA, 19 bp, Artificial Sequence
  c-FOS shRNA
tccgaagagaacggaataa

SEQ ID NO: 5 - DNA, 19 bp, Artificial Sequence
  scramble shRNA
gttattactgttcgatcgc

(19) United States
(12) Patent Application Publication
(10) Pub. No.: US 2016/0074467 A1
(43) Pub. Date: Mar. 17, 2016

(54) TREATMENT OF AGING EFFECTS BY GONADOTROPIN-RELEASING HORMONE, NEUROGENESIS OR BRAIN IKK BETA/NF-KAPPAB INHIBITION

(71) Applicant: ALBERT EINSTEIN COLLEGE OF MEDICINE, INC., Bronx, NY (US)

(72) Inventor: Dongsheng Cai, Larchmont, NY (US)

(73) Assignee: ALBERT EINSTEIN COLLEGE OF MEDICINE, INC., Bronx, NY (US)

(21) Appl. No.: 14/787,278

(22) PCT Filed: Apr. 24, 2014
(86) PCT No.: PCT/US14/35220

§ 371 Date: Oct. 27, 2015

Related U.S. Application Data
(60) Provisional application No. 61/817,401, filed on Apr. 30, 2013.

(51) Int. Cl. A61K 38/09; A61K 49/00

(52) U.S. Cl. CPC A61K 38/09; A61K 49/0008

(57) ABSTRACT

Methods are disclosed for treating effects of aging using gonadotropin-releasing hormone, agents that inhibit IκB kinase-β (IKK-β) and/or nuclear factor κB (NF-κB), and/or agents that promote neurogenesis.

### CROSS-REFERENCE TO RELATED APPLICATIONS

This application claims the benefit of U.S. Provisional Patent Application No. 61/817,401, filed on Apr. 30, 2013, the content of which is incorporated by reference.

### STATEMENT OF GOVERNMENT SUPPORT

This invention was made with government support under grant numbers R01 AG031774 and R01 DK078750 awarded by the National Institutes of Health. The government has certain rights in the invention.

### BACKGROUND OF THE INVENTION

Throughout this application various publications are referred to in superscript. Full citations for these references may be found at the end of the specification before the claims. The disclosures of these publications are hereby incorporated by reference in their entireties into the subject application to more fully describe the art to which the subject application pertains.

Aging is characterized by the gradual and overall loss of various physiological functions, leading to the end of lifespan. Although the search for resolution of aging pathology is continuing^{1 6}, research has shown that certain neurons can mediate environmental influences on aging in Caenorhabditis elegans and Drosophila, and neural manipulations of insulin or insulin-like growth factor 1 signalling or uncoupling protein 2 were shown to affect lifespan in animals⁷⁻¹¹. The present invention addresses the need to treat the effects of aging in the elderly.

### SUMMARY OF THE INVENTION

The invention provides methods of treating an aging effect in a subject comprising administering to the subject gonadotropin-releasing hormone (GnRH) in an amount that is effective to treat an aging effect in a subject.

The invention also provides methods of treating an aging effect in a subject comprising promoting neurogenesis in the brain of the subject in an amount that is effective to treat an aging effect in a subject.

The invention further provides methods of screening for a candidate agent that treats an aging effect in a subject comprising determining whether or not the agent inhibits IκB kinase-β (IKK-β) and/or nuclear factor κB (NF-κB) in the brain, wherein an agent that inhibits IKK-β and/or NF-κB is a candidate agent for treating an aging effect in a subject.

### BRIEF DESCRIPTION OF THE DRAWINGS

FIG. 1A-1D. Aging-dependent hypothalamic NF-κB activation. C57BL/6 mice (chow-fed males) were analyzed at young (3-4 months) age (Y), middle-old (11-13 months) age (M), and old (22-24 months) age (O). a, b, Hypothalamus and cortex were analyzed via western blots. b, The intensity of phosphorylated RelA (p-RelA) normalized to RelA in hypothalamus (HYT) and cortex. AU, arbitrary units. c, d, Mice received MBH injections of lentiviral GFP controlled by NF-κB response element (NF-κB/RE), and after a ˜3-week recovery, brain sections were made to reveal GFP and NeuN staining. 4′,6-diamidino-2-phenylindole (DAPI) staining shows entire cell populations. Scale bar, 25 μm. c, Images of MBH subregion. d, Percentages of cells co-expressing GFP and NeuN (GFP⁺ NeuN⁺) among NeuN-expressing cells (NeuN⁺) in the MBH. **P<0.01; ***P<0.001; n=6 (b) and 3 (d) per group. Error bars reflect mean±s.e.m.

FIG. 2A-2G. Aging manipulations by hypothalamic IKK-β and NF-κB. MBH-IKK-β, MBH-IκB-α and MBH-ctrl mice were generated using ˜18-month-old C57BL/6 mice (chow-fed males) via MBH injections of lentiviruses expressing ^{CA}IKK-β, ^{DN}IκB-α and control GFP under the control of synapsin promoter. a, Lifespan of these mice (n=23-31 mice per group). b, c, Mice at ˜6 months after gene delivery were assessed for cognition via the T-maze (b) and muscle endurance (c). d-g, Mice were killed at 8-10 months after gene delivery for measuring muscle (quadriceps) fiber size (d), dermal thickness (e), bone mass (f), and tail tendon breaking time (g). *P<0.05; **P<0.01; MBH-ctrl: n=23 (a), 9 (b), 6 (c), 3 (d, e), 4 (f) and 7 (g); MBH-IKK-β: n=24 (a), 10 (b), 6 (c), 3 (d, e), 4 (f) and 5 (g); MBH-IκB-α: n=31 (a), 12 (b), 7 (c), 3 (d, e), 6 (f) and 8 (g). Error bars reflect mean±s.e.m.

FIG. 3A-3I. Role of hypothalamic microglia in aging. a-c, Brain sections of young, middle-old and old C57BL/6 mice were analyzed for hypothalamic microglia. a, Images of immunostaining in MBH subregion. Scale bar, 25 μm. b, c, Numbers of cells expressing Iba-1 (Iba-1⁺) (b) or TNF-α (TNF-α⁺) (c) in the hypothalamic medial basal region (across the confocal microscopic field of serial sections under ×200 magnification). d, Middle-old Ikbkb^{lox/lox} mice received bilateral MBH injections of lentiviral CD11b promoter-driven Cre (CD11b) versus control (ctrl). At 1 or 8 months after injection, brain sections were made for Iba-1 and TNF-staining. Mice generated at a young age provided normal references. Data show numbers of cells immunoreactive for Iba-1, TNF-α or both in the arcuate nucleus. e-i, Mice were generated via viral injections at a middleold age and assessed at old ages for cognition (e-g), muscle endurance (h), and tail tendon breaking time (i). Morris water maze data included time in target quadrant versus one representative off-target (off-T) quadrant in probe trials. *P<0.05; **P<0.01; ***P<0.001; n=4 (b, c) and 3 (d) per group; ctrl: n=6 (e-g, i) and 9 (h); CD11b: n=5 (e-g) and 6 (h, i). Error bars reflect mean±s.e.m.

FIG. 4A-4I. Genetic longevity by brain-specific IKK-β knockout. N/Ikbkb^{lox/lox} mice (N/Ikbkb^{l/l}) and wild-type littermates (WT) males were maintained on chow since weaning. a, b, Young (3 months) and old-age (18-20 months) mice were tested for cognition (a) and muscle endurance (b). Morris water maze data included time in target northwest (NW) versus off-target northeast (NE), southwest (SW) and southeast (SE) quadrants in probe trials. c-h, Young (3-4 months) and old (20-24 months) mice were killed for assessing muscle (quadriceps) fiber size (c), dermal thickness (d-f), bone mass (g), and tail tendon breaking time (h). i, Lifespan follow-up (n=20 in wild type and n=25 in N/Ikbkb^{l/l}). *P<0.05; **P<0.01; young wild type: n=10 (b), 3 (c, e), 5 (f), 6 (g) and 8 (h); young N/Ikbkb^{l/l}: n=14 (b), 3 (c, e, f), 6 (g) and 8 (h); old wild type: n=10 (a), 7 (b), 3 (c, e), 5 (f, g) and 6 (h); old N/Ikbkb^{l/l}: n=10 (a), 7 (b), 3 (c, e, f) and 6 (g, h). Error bars reflect mean±s.e.m.

FIG. 5A-5J. Inhibition of GnRH by IKK-β and NF-κB. a-c, Hypothalamic Gnrh1 mRNA of mice which are described in FIGS. 2 and 3. d-g, GT1-7 cells were transfected with ^{CA}IKKβ, RelA or ^{DN}IkBα or control plasmid (d, e, g), co-transfected with Gnrh1-promoter luciferase plasmid (e, f), or together with Rela short hairpin RNA (shRNA) (sh-RelA) or control shRNA (sh-ctrl) plasmid (f), and were measured for GnRH release (d), Gnrh1 promoter (e, f), and Fos, Jun, Prkca and Prkcd mRNA levels (g). h, Gnrh1 promoter activities were measured for GT1-7 cells transfected with Gnrh1-promoter luciferase plasmid, co-transfected with Jun or Fos plasmid versus control plasmid, or treated with 12-O-tetradecanoylphorbol-13-acetate (TPA) or vehicle (veh). i, Gnrh1 promoter activities were measured for GT1-7 cells transfected with Gnrh1-promoter luciferase plasmid, co-transfected with ^{CA}IKKβ or control plasmid, and with Fos and Jun shRNA plasmids (sh-c-Fos and sh-c-Jun) or scramble shRNA control (sh-ctrl). j, Summarized schematic model. *P<0.05, **P<0.01; ***P<0.001; n=12 (a, e) and 3 (f-i) per group, and n=6 (b), 8 (c) and 4 (d) in control, n=8 (b) and 6 (d) in IKK-β, and n=8 (c) and 6 (d) in IκB-α. Error bars reflect mean±s.e.m.

FIG. 6A-6I. Central and systemic actions of GnRH in counteracting aging. a-d, C57BL/6 mice at an old age were subjected to neurogenesis (a, b) and survival (c, d) assays, as detailed in the Methods. a, BrdU staining images of MBH subregion and dentate gyrus (DG) in neurogenesis assay. Scale bar, 50 nm. b, c, BrdU-labelled (BrdU⁺) cells in the MBH (b) and dentate gyrus (c) in a neurogenesis assay. d, e, Survival of BrdU-labelled (BrdU⁺) cells in the MBH (d) and dentate gyrus (e) in a survival assay. f-i, MBH-IKK-β and MBH-ctrl mice at an old age were daily injected subcutaneously with GnRH or vehicle for 5 weeks, and analyzed for muscle endurance (f), skeletal muscle fibers (g), dermal thickness (h) and cognition (i). *P<0.05; **P<0.01; ***P<0.001; n=4 (b-e), 7 (f) and 3 (g, h) per group, and n=12 (control, vehicle), 7 (control, GnRH), 7 (IKK-β, vehicle) and 8 (IKK-β, GnRH) (i). Error bars reflect mean±s.e.m.

FIG. 7A-7H. Aging retardation by hypothalamic NF-κB inhibition in females. Female C57BL/6 mice (chow-fed, ˜14-month-old) received MBH injections of lentiviral DNIκB-α (IκB-α) vs. control (Ctrl), as described in FIG. 2. At ˜4 months post gene delivery, following assessment of technical qualification via swimming speed test (a), mice were examined with Morris Water Maze (MWM) showing the performance during the training session (b) and probe trials (c&d). Subsequently, mice were assessed for muscle endurance (e), muscle fiber sizes (f), skin thickness (g), and bone mass (h). *P<0.05, **P<0.01, Ctrl: n=8 (a-d), 6 (e), 3 (f&g), and 5 (h); IκB-α: n=6 (a-e, h), and 3 (f&g). Error bars reflect mean±SEM.

FIG. 8A-8K. Aging retardation in female N/Ikbkb^{lox/lox} mice. Female N/Ikbkb^{lox/lox} mice (N/Ikbkb^{l/l}) and littermate WT (Ikbkblox/lox background) were maintained on a normal chow since weaning. At an old age, after the assessment of technical qualification via open field (a&b) and swimming speed test (c), mice were assessed for cognition via Morris Water Maze (MWM) (d-f), muscle endurance (g), muscle fibers (h), skin (i), bone mass (j), and tail tendon breaking time (k). MWM results show performance during training session (d), and time distribution in target vs. a representative off-target quadrant (e) and latency (f) during subsequent probe trials. *P<0.05, **P<0.01; WT: n=11 (a-c, g), 8 (d-f), 3 (h&i), and 6 (j&k); N/Ikbkbl/l: n=9 (a-g), 3 (h&j), 6 (j), and 4 (k). Error bars reflect mean±SEM.

### DETAILED DESCRIPTION OF THE INVENTION

The invention provides a method of treating an aging effect in a subject comprising administering to the subject gonadotropin-releasing hormone (GnRH) or an analog thereof in an amount that is effective to treat an aging effect in a subject.

GnRH or an analog thereof can be administered to the central nervous system or to the cerebral spinal fluid of the subject. Alternatively, GNRH or an analog thereof can be administered systemically to the subject. GnRH or an analog thereof can be coupled to an agent that increases its penetration across the blood-brain barrier.

The invention also provides a method of treating an aging effect in a subject comprising promoting neurogenesis in the brain of the subject in an amount that is effective to treat an aging effect in a subject. Neurogenesis can be promoted, for example, through central or systemic injection of a pharmacologic agent, delivery of a gene through a delivery vector, or delivery of stem cells in a brain region or a brain ventricle. Preferably, neurogenesis is promoted in the hypothalamus of the subject, as well as in the hippocampus, supra-ventricular zone and/or other regions of the brain of the subject.

The invention further provides a method of screening for a candidate agent that treats an aging effect in a subject comprising determining whether or not the agent inhibits IκB kinase-β (IKK-β) and/or nuclear factor κB (NF-κB) in the brain, wherein an agent that inhibits IKK-β and/or NF-κB is a candidate agent for treating an aging effect in a subject. The screening can be carried out, for example, using a middle aged (e.g., around 1-1.5 years old) mouse or an elderly (e.g., older than 1.5 years) mouse. For example, as described herein below, to visualize NF-κB activity in the brain directly, a NF-κB reporter can be used that induces green fluorescent protein (GFP) after the binding of NF-κB to its transcriptional response element in a lentiviral vector. Preferably, IKK-β and/or NF-κB is inhibited in the hypothalamus, hippocampus, and/or supra-ventricular zone of the brain.

In any of the methods disclosed herein, the aging effect can comprise, for example, one or more of cognitive decline, muscle loss and/or muscle weakness, and loss of bone mass. As used herein, to treat an aging effect means to reduce the progression of an aging effect, for example, to reduce cognitive decline, muscle loss and/or muscle weakness, and/or loss of bone mass that occurs with aging.

The subject being treated can be a middle-aged subject. For example, the middle-aged subject can be a human subject who is at least 30 years of age. The subject being treated can also be an elderly subject. For example, the elderly subject can be a human subject who is at least 60 or 65 years of age.

It is noted that mice and humans are reported to have an maximum life-span potential of 4 years and 120 years, respectively.⁴³ Thus, a 60 year old human may correlate roughly with a 2 year old mouse, and a 30 year old human may correlate roughly with a 1 year old mouse. Similarly, in rats, it has been reported that in adults, every month of age for a rat is approximately equivalent to 2.5 human years.⁴⁴

The compound or agent can be administered to the subject in a pharmaceutical composition comprising a pharmaceutically acceptable carrier. Examples of acceptable pharmaceutical carriers include, but are not limited to, additive solution-3 (AS-3), saline, phosphate buffered saline, Ringer's solution, lactated Ringer's solution, Locke-Ringer's solution, Krebs Ringer's solution, Hartmann's balanced saline solution, and heparinized sodium citrate acid dextrose solution. The pharmaceutically acceptable carrier used can depend on the route of administration. The pharmaceutical composition can be formulated for administration by any method known in the art, including but not limited to, oral administration, parenteral administration, intravenous administration, transdermal administration, intranasal administration, administration through an osmotic mini-pump, and administration directly to the brain or cerebral spinal fluid.

This invention will be better understood from the Experimental Details that follow. However, one skilled in the art will readily appreciate that the specific methods and results discussed are merely illustrative of the invention as described more fully in the claims that follow thereafter.

### Experimental Details

### Introduction

This study focused on the hypothalamus, a key brain region that is crucial for the neuroendocrine interaction between the central nervous system and the periphery, and investigated whether the hypothalamus may have a fundamental role in aging development and lifespan control, in addition to its critical involvement in basic life-supporting functions such as growth, reproduction and metabolism. An atypical collection of hypothalamic inflammatory changes can broadly and causally underlie the development of metabolic syndrome components including being overweight, glucose intolerance and hypertension12-15, and of note, all of these disorders are often related to aging. Furthermore, microglia are involved in neurodegenerative diseases¹⁶⁻²⁴, which aligns with the appreciated connection between systemic immunity and aging^{25,26}. Here, through targeting hypothalamic immunity/inflammation, it was demonstrated that the hypothalamus is fundamentally important for aging and lifespan control.

### Materials and Methods

Summary. All mice in this study were in C57BL/6 background, and Ikbkb^{lox/lox} and nestin-Cre mice were described previously¹³. Physiological analyses included open field, visual platform test, Morris water maze, T-maze and grip test. Skin and muscle histology, bone mass via X-ray microtomography, and tail tendon breaking time were examined using standard methods in the literature. Lentiviral DNAs, virus production, MBH injection, immunostaining, western blot, and real-time PCR were similarly used in recent research¹³. The Gnrh1 promoter was analyzed in GT1-7 cells transfected with Gnrh1 promoter-driven luciferase plasmids. Statistics included analysis of variance (ANOVA) and appropriate post-hoc analyses for comparisons involving more than two groups and two-tailed Student's t-test for comparisons involving only two groups. Data were presented as mean±s.e.m. P<0.05 was considered significant.

Mouse models and treatments. Nestin-Cre mice and Ikbkb^{lox/lox} mice were described in previous publications^{13-15,35}, and maintained on C57BL/6 strain for more than 15 generations. C57BL/6 mice were obtained from Jackson Laboratory or the National Institute of Aging, NIH. All mice were kept under standard and infection-free housing, with 12-h light/12-h dark cycles and 4-5 mice per cage. Pathogen-free quality was ensured with quarterly serology, quarterly histopathological examinations and routine veterinarian monitoring, and a bacteriological test was additionally included. All mice in this study were maintained on a normal chow from LabDiet (4.07 kcal g⁻¹).

For animal GnRH therapy, mice were subcutaneously injected with GnRH (Sigma) at the dose of 2 ng per mouse on a daily basis for a period of 5-8 weeks. The Institutional Animal Care and Use Committee at the Albert Einstein College of Medicine approved all the procedures. Body weight and food intake were measured regularly using a laboratory scale. The grip test was performed to measure muscle endurance using the method as similarly described in the literature^{36,37}, using a homemade square grid with a small mesh size to allow mice to hang for longer time. In brief, a mouse was lifted by the tail and placed on a homemade square grid (1-cm mesh size). The grid was then inverted 30.5 cm over a soft pad, and the mouse was allowed to hang by paws for a maximum of 5 min. The time that the mouse was able to hang was recorded during a 5-min test period.

Lentiviruses and MBH injection. Synapsin promoter-directed lentiviral vector was used to drive neuron-specific gene delivery as previously established^{13-15,35}. These lentiviral vectors contain the cDNA of ^{CA}IKK-β or ^{DN}IκB-α or only GFP controlled by synapsin promoter. To create lentiviral NF-κB reporter vector, a target plasmid was constructed to have the GFP open reading frame controlled by a DNA cassette containing five tandem repeats of the NF-κB transcriptional response element, according to the approach established in the literature³⁸. The lentiviruses were produced from HEK293T cells via co-transfecting a target plasmid with two package plasmids (VSVg and delta 8.9) using CaCl₂. Lentiviruses were purified through ultracentrifugation. Intra-MBH viral injections were performed as we previously established^{13-15,35}. In brief, under an ultraprecise stereotactic instrument (resolution: 10 μm) (Kopf Instruments), lentiviruses were bilaterally injected at the coordinates of 1.5 mm posterior to the bregma, 5.8 mm below the skull, and 0.2 mm lateral to the midline.

Cognitive behavioral tests. All mice were tested for general health, sensorimotor reflexes and motor responses before the onset of all behavioural testing. Mice were maintained on a 12-h light/12-h dark schedule in an isolation unit located inside the behavioural testing room. An Anymaze video tracking system (Stoelting) equipped with a digital camera connected to a computer was used to videotape the whole course of animal activities in training and experimental sessions of behavioural tests.

Open field test. Locomotor activities were assessed using the open field test. The open field arena consisted of a clear Plexiglas chamber that was 40 cm×40 cm, with walls that were 35 cm high. The arena was placed in a brown box to reduce visual cues. Mice were placed in the arena and allowed to explore for 5 min, and measured for distance and time travelled and mean speed.

Morris water maze test. The maze was filled with 22-23° C. water that was made opaque with Crayola non-toxic paint, and was located in the centre of a small square room with numerous extra-maze cues (various black shapes on white background, a cabinet and an experimenter). The diameter of the maze was 90 cm and divided into four quadrants (northwest, northeast, southwest and southeast). A circular platform with a diameter of 10 cm was placed 25 cm from the wall in the centre of the northwest quadrant.

Visual platform test. The visual platform test was performed on a single day. There were six trials with 30-min inter-trial intervals. In the test, a visible flag was placed on the top of the platform to increase the visibility, and the platform was placed on a random location for each trial. A mouse was placed on water, at the same starting location for all trials, and was measured for latency, distance and mean speed travelled to the platform.

Hidden-platform training. Mice were first required to swim to and sit on a circular visible platform at 0.5 cm above water level for 10 s. If mice could not find the platform within 60 s, they were gently guided to the platform using a glass stirring rod. Mice were then subjected to five consecutive days of training, consisting of two trials per entry location (entry locations were north, south, east and west) for a total of eight trials per day. The platform was made invisible by submerging it 1 cm below the surface of the water. Mice were expected to find the location of the invisible platform, and measured for latency to reach the platform, distance travelled to reach the platform, path efficiency, time spent in and distance travelled in each quadrant as well as total distance and mean swim speed.

Probe trial. On day 6, mice were subjected to a single probe trial, in which the platform was removed and mice were allowed to swim for 60 s. Mice were measured for the amount of time spent in all quadrants, distance and number of times that mice crossed the location of the former platform, and total distance and mean swim speed.

T-maze. Mice were tested for reward (1:1 water/full-fat sweetened condensed milk) (Nestle) on a forced-choice alternation test in a T-maze with an opaque floor and plastic sides. Mice first received food restriction to reduce body weight by 5-10%, and then a 4-day adaptation to the apparatus with the reward. After that, mice were given six pairs of training per day for 12 days, and tests of every 2 days were designated as a trial block. On the first trial of each pair, a mouse was placed in the start arm, forced to choose one of two goal arms in the T (the other is blocked by a removable door), and received the reward at the end. The mouse was kept in this goal arm for 15-20 s and subsequently returned by the experimenter to the start arm. The animal was then given a free choice between two goal arms, rewarded for choosing the ‘novel’ arm (the one that was not chosen in the first trial of the pair), but punished for choosing the other goal arm (the one that was chosen on the first trial of the pair) using a 20 s-blocking without the reward. The location of the sample arm (left or right) was varied across trials so that mice received equal numbers of left and right presentations, and no more than two consecutive trials with the same sample location. Mice were tested in squads of 4-5 to minimize variations in inter-trial intervals, which was 5-10 min for all animals throughout 12-day training period.

Ex vivo analyses. Collagen cross-linking. The method of tail tendon breaking test was used to examine collagen cross-linking, as described³⁹. In brief, a collagen fiber was teased from a mid-tail section of the lateral tail tendon and tied to a 2-g weight. The fiber was suspended into a bath containing 7 M urea at 45° C. The fiber breaking time was determined in quadruplicate for each mouse.

Tissue histology. Skeletal muscles (quadriceps) and dorsal skin were dissected from mice, fixed in 10% neutralized formalin at 4° C. overnight, and embedded into paraffin. Paraffin sections were prepared at 5-μm thickness and subjected to haematoxylin and eosin staining. Images were collected using an Axioskop II light microscope (Zeiss) and analyzed using Image J.

Bone volume fraction measurement. The bone volume fraction procedure established in the literature⁴⁰ was adopted. In brief, the left intact femurs were removed and analyzed via LaTheta LCT-100A X-ray microtomography scanner (Aloka) through mouse physiology core facility at Albert Einstein College of Medicine. The distal part of femur encompassing the cancellous bone was analyzed. The trabecular and cortical bone regions were outlined for each tomography slice by the software of the scan system. Bone volume fraction was calculated as the trabecular bone volume divided by the total bone volume. A calibration phantom was used for calibration of each scan.

Immunostaining, histology and western blot. Mice under anaesthesia were perfused with 4% paraformaldehyde, and brains were removed, post-fixed in 4% paraformaldehyde, and infiltrated in 20-30% sucrose. Brain sections were made at 10-μm thickness via a cryostat, blocked with serum, penetrated with Triton-X 100, treated overnight at 4° C. with primary antibody, followed by reaction with fluorescence-conjugated secondary antibody (Jackson), and imaged under a confocal microscope. For BrdU staining, sections were pre-treated with 1 M HCl for 30 min at 37° C., followed by 5-min treatment with 0.1 M sodium borate, pH 8.5. Primary antibodies included rabbit anti-Iba-1 (Wako), rabbit anti-GFAP (Millipore), mouse anti-TNF-α (Abcam), mouse anti-NeuN (Millipore), and goat anti-Cre antibody (Santa Cruz). For Nissl staining, freshly isolated mice brains were fixed in 4% paraformaldehyde in PBS for overnight at 4° C. The fixed whole brains were then subjected to cryosectioning coronally, and frozen sections were stained to detect Nissl body by using the NovaUltra Nissl stain kit (IHCWORLD) according to the manufacturer's instruction.

Serial brain sections across the MBH were made at 20 μm thickness, and every five sections were represented by one section with staining and cell counting. For western blotting, animal tissues were homogenized, and proteins were dissolved in a lysis buffer, and western blots were conducted as previously described³. Proteins separated by SDSPAGE were identified by immunoblotting with primary rabbit anti-pRelA, anti-RelA and anti-β-actin antibodies (Cell Signaling) and horseradish peroxidase (HRP)-conjugated anti-rabbit secondary antibody (Pierce).

DNA vectors, cell culture and molecular/biochemical analysis. Promoter sequence of the rat Gnrh1 gene was PCR amplified (−1934 to +21) from a rat genomic DNA preparation, and subcloned into the pGL3-basic luciferase reporter vector (Promega) using standard cloning strategies. pcDNA expressing ^{CA}IKK-β or ^{DN}IκB-α versus control were previously described^{13-15,35}, or pcDNA expressing HA-RelA was provided by A. Lin. RelA shRNA and control (GFP) shRNA vectors were obtained from Addgene, as studied in the literature. RelA shRNA: 5′-GCATGCGATTCCGCTATAA-3′ (SEQ ID NO:1); control shRNA: 5′-ACAGCCACAACGTCTATAT-3′ (SEQ ID NO:2). Expression plasmids for c-Jun or c-Fos were provided by D. Stocco. Vectors expressing c-Jun or c-Fos shRNA or scramble shRNA control were provided by L. Fahana. c-Jun shRNA: 5′-AGTCATGAACCACGTTAAC-3′ (SEQ ID NO:3); c-Fos shRNA: 5′-TCCGAAGAGAACGGAATAA-3′ (SEQ ID NO:4); scramble shRNA: 5′-GTTATTACTGTTCGATCGC-3′ (SEQ ID NO:5). 12-O-tetradecanoylphorbol-13-acetate (also known as phorbol 12-myristate 13-acetate or TPA) and calphostin-C were from Sigma-Aldrich. TPA or calphostin-C was dissolved in dimethylsulphoxide (DMSO) and applied in cell culture medium at a final concentration of 0.2 μM or 0.01 μM, respectively, and DMSO did not exceed 0.1% of cell culture medium. GT1-7 cells were previously described¹³, and cultured in a standard humidified incubator at 37° C. and 5% CO₂ with DMEM cell culture medium supplemented with 10% FBS, 2 mM L-glutamine, and PenStrep (50 U ml⁻¹ penicillin G, 50 μg ml⁻¹ streptomycin). Transfection of cultured cells with luciferase plasmids and expression plasmids was performed through Lipofectamine 2000 (Invitrogen). The dual luciferase reporter assay (Promega) was performed according to the manufacturer's instruction, and co-transfection of the pRL-TK vector expressing Renilla luciferase was used to control firefly activity internally. Empty plasmids pGL3 and pcDNA3.1 were used as negative controls. RNA was extracted by TRIzol (Invitrogen) and analyzed via SYBR green real-time PCR (StepOnePlus real-time PCR system, Invitrogen). Testosterone and oestradiol were measured using testosterone and oestradiol EIA kits (Cayman Chemical). GnRH was measured using the luteinizing hormone-releasing hormone EIA kit (Phoenix Pharmaceuticals).

BrdU labelling study. Mice were pre-implanted with intracerebroventricular (i.c.v.) cannula in the hypothalamic third ventricle, and after a ˜3-week recovery, they were subjected to neurogenesis assay or survival-assays. In the neurogenesis assay, mice were daily pre-injected with GnRH or vehicle at the dose of 1 ng per day through cannula for 3 days, subsequently a single i.c.v. injection of BrdU (Sigma) at the dose of 10 μg (defined as day 0), and continued to receive daily i.c.v. injections of GnRH (1 ng per day) or vehicle for 7 days before they were killed for brain sectioning. In the survival-assay, mice pre-received daily i.c.v. injections of GnRH (1 ng per day) or vehicle for 3 days, then daily i.c.v. injections of BrdU (10 μg per day) together with daily i.c.v. injections of GnRH (1 ng per day) or vehicle for 7 days (last day was defined as day 7), and followed by continued daily i.c.v. injections of GnRH (1 ng per day) or vehicle until day 30 when mice were killed for brain sectioning.

Statistical analyses. Kolmogorov-Smirnov test was used to determine parametric distribution of data. Analysis of variance (ANOVA) and appropriate post-hoc analyses were used for comparisons involving more than two groups. Two-tailed Student's t-tests were used for comparisons involving only two groups. Lifespan analysis was performed using Kaplan-Meier survival analysis; the mutant and control survivorship curves were compared in pairs and P values were obtained with log-rank test. Maximal lifespan of mice were statistically analyzed using Chi-squared test according to the literature⁴². All data were presented as mean±s.e.m. P<0.05 was considered significant.

### Results

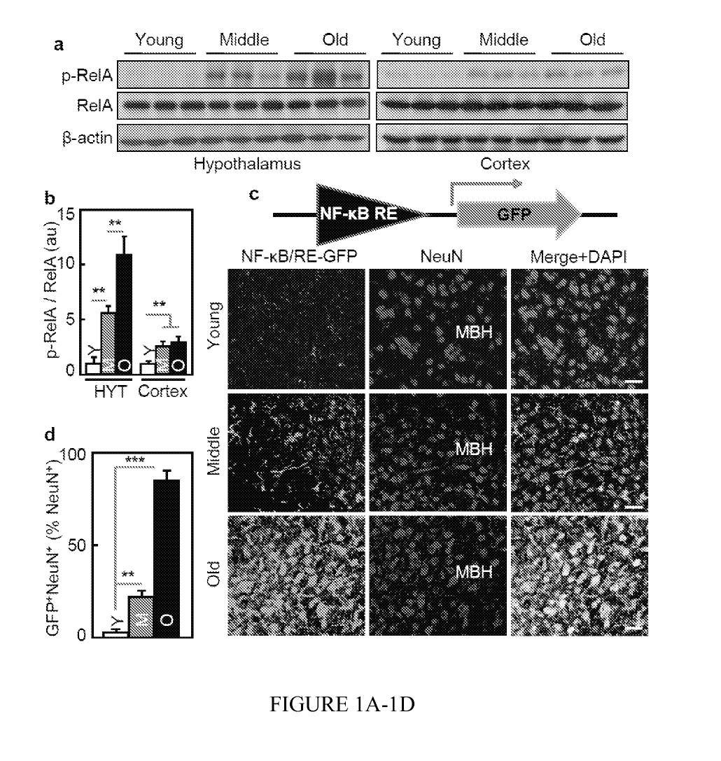

Aging-dependent hypothalamic NF-κB activation. In studying the role of the hypothalamus in aging, a strategy of targeting hypothalamic immunity was developed. As shown in recent work¹²⁻¹⁵, infection-unrelated inflammatory changes in the mediobasal hypothalamus (MBH) contribute to the development of various metabolic syndrome components, and the molecular basis is mediated crucially by NF-κB and its upstream IKK-β. Indeed, using phosphorylation of NF-κB subunit RelA to report NF-κB activation, although hypothalamic NF-κB was barely active in mice of young age (3-4 months), it was activated in the hypothalamus of mice at middleold ages (11-13 months), and the activities further increased as the mice became older (22-24 months) (FIGS. 1a, b). Agreeing with this observation, messenger RNA levels of many cytokines and immune regulators increased in the hypothalamus of old mice compared to the young group (data not shown). To visualize NF-κB activity in the MBH directly, a NF-κB reporter was used that induces green fluorescent protein (GFP) after the binding of NF-κB to its transcriptional response element in a lentiviral vector (FIG. 1c). After in vitro assessment of this approach (data not shown), animal experiments were performed by delivering this lentiviral NF-κB reporter into the MBH of mice at young, middleold and old ages. A prolonged recovery period was used to minimize the procedure-related nonspecific effects on NF-κB. GFP was negligible in the MBH of young mice (FIG. 1c), but was evident in the MBH of middleold mice and became more profound in old mice (FIGS. 1c, d), confirming that aging is associated with hypothalamic NF-κB activation. This lentiviral NF-κB reporter was also injected into various other brain regions. Comparatively, the MBH was most sensitive to aging-related NF-κB activation (data not shown). Of interest, immunostaining with the neuronal marker NeuN revealed that NF-κB activation in neurons was relatively modest under middleold aging, but became prominent when age further increased (FIGS. 1c, d). Thus, aging development is characterized by chronic activation of NF-κB-directed innate immune pathway predominantly in the hypothalamus.

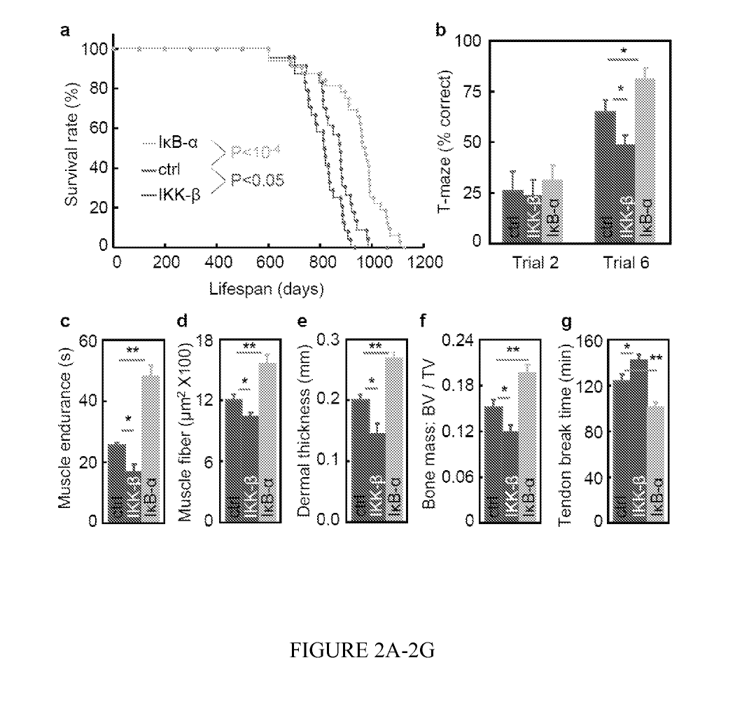

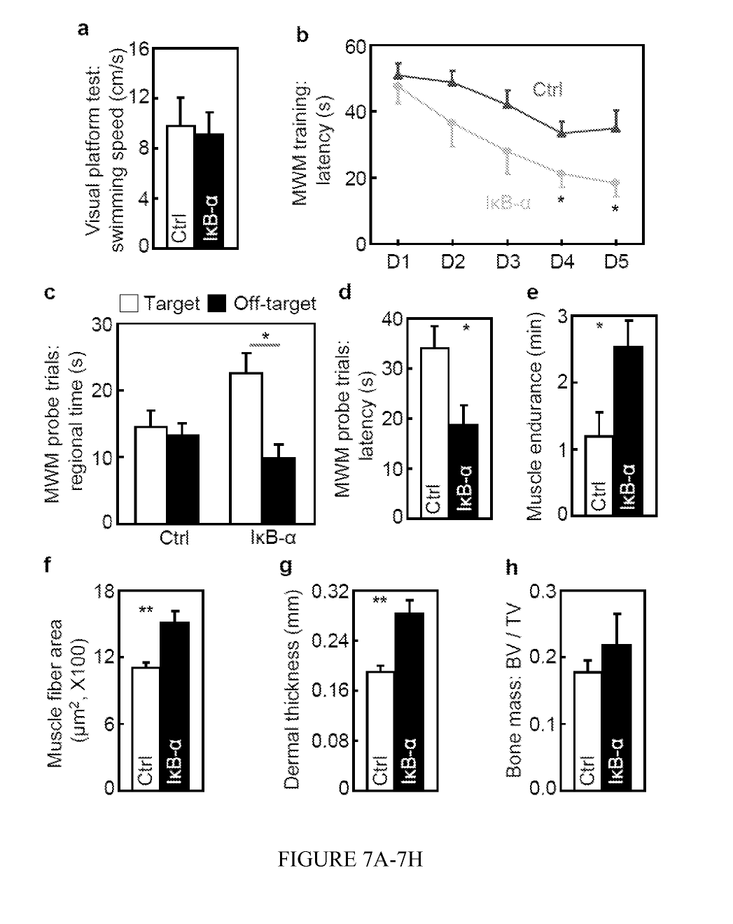

Control of Aging by hypothalamic IKK-β and NF-κB. The proposed involvement of IKK-β and NF-κB in the hypothalamic control of aging was then tested with focus on the MBH. Using MBH-directed lentiviral gene delivery as previously established^{12,13}, the dominant-negative IκB-α (^{DN}IκB-α) was delivered to inhibit NF-κB, and constitutively active IKK-β (^{CA}IKK-β) was used to activate NF-κB in MBH neurons. MBH delivery of GFP in the same lentiviral system was used as the control (data not shown). Middleold C57BL/6 mice received bilateral MBH lentiviral injections; use of middleold age mice helped to eliminate developmental concerns, and, indeed, aging retardation can be achieved through intervention starting at a middleold age²⁷. These mice with MBH delivery of ^{DN}IκB-α, ^{CA}IKK-β and control GFP were named MBH-IκB-α, MBH-IKK-β and MBH-ctrl mice, respectively, and all mice were maintained under pair feeding of a normal chow so that they had similar daily food intake. Longitudinal follow-up revealed that MBH-ctrl mice displayed a typical pattern of lifespan (FIG. 2a), which indicated that the approach of MBH injection was technically suitable. Importantly, lifespan significantly increased in MBH-IκB-α mice but decreased in MBH-IKK-β mice compared to controls (FIG. 2a). In parallel with lifespan analysis, separate mice were generated to evaluate aging-related physiology and histology. Cognition and muscle endurance of mice were assessed at ˜6 months after gene delivery, at which hypothalamic NF-κB remained overactivated in MBH-IKK-β mice but suppressed in MBH-IκB-α mice (data not shown). In cognitive tests, compared to controls, MBH-IκB-α mice performed better but MBH-IKK-β mice performed worse (FIG. 2b), and all of these mice were technically eligible for the test (data not shown). These mice were also subjected to a grip test, showing that aging-related muscle weakness was attenuated in MBH IκB-α mice but worsened in MBH-IKK-β mice (FIG. 2c). Furthermore, these mice were examined for a panel of histological biomarkers including muscle size, skin thickness, bone mass, and tail tendon collagen cross-linking. As shown in FIG. 2d-g, aging-related changes of these biomarkers were dampened in MBH-IκB-α mice but exacerbated in MBH-IKK-β mice. Finally, given that these data were based on males, female mouse models were generated. Results from females agreed with the observations in males (FIG. 7). In sum, the hypothalamus has a unique role in the development of systemic aging, and hypothalamic IKK-β and NF-κB represents a driving force in this process.

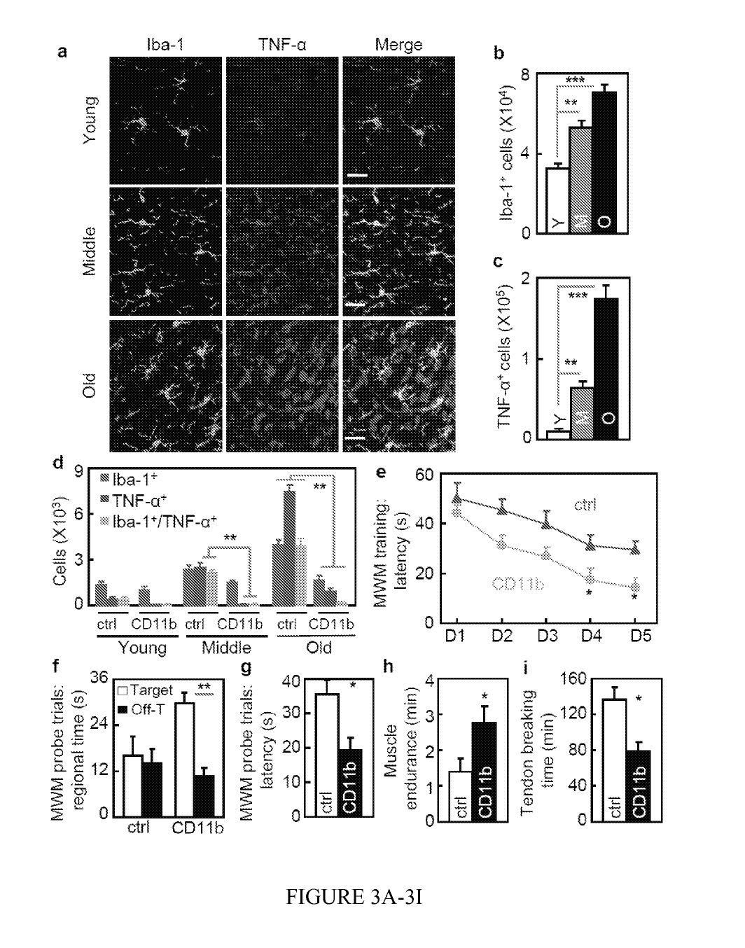

Hypothalamic microglia in aging development. To understand aging-related hypothalamic immunity/inflammation further, microglia were profiled in the hypothalamus. Using immunostaining, the numbers of microglial cells in the MBH were found to increase in an age-dependent manner (FIGS. 3a, b). Overproduction of tumour necrosis factor-α (TNF-α) (FIGS. 3a, c) and activation of NF-κB (data not shown) were both detected in these microglial cells, indicating that they were inflammatory. Under early aging, NF-κB activation was already evident in hypothalamic microglia; however, this change was still modest in hypothalamic neurons (FIGS. 1c, d). Also as observed, TNF-α overproduction was mostly limited to hypothalamic microglia during early aging, but became prevalent across the MBH, which affected other neural cells (such as neurons) in this region. Hypothalamic Tnfα mRNA levels were measured in mice of different ages; data obtained (not shown) correlated well with cell counting of TNF-α immunostaining (FIG. 3c). It should be mentioned that TNF-α is a gene product of NF-κB and also acts to activate IKK-β and NF-κB. Overall, the data indicate that TNF-α is generated mainly by microglia during early aging, and the paracrine actions of this cytokine on neighbouring cells is predicted to lead to aging-associated neuronal IKKβ and NF-κB activation. In the literature, TNF-α is known to be neurotoxic or neuroprotective²⁸⁻³⁰, which may reflect the differential functions of soluble versus transmembrane TNF-α³⁰. In the aging model, soluble TNF-α seems to be involved in IKK-β and NF-κB-mediated microglianeuron crosstalk that controls systemic aging.

Hypothalamic control of aging by microglial IKK-β. Subsequently, a mouse model was generated with IKK-β knockout in the MBH microglia through bilaterally delivering microglia-specific (CD11b promoter-driven) lentiviral Cre into the MBH of Ikbkb^{lox/lox} mice. Control Ikbkb^{lox/lox} mice were injected with Cre-deficient lentiviruses. Cre was delivered specifically in ionized calcium binding adaptor molecule 1 (Iba-1)-expressing microglia, and most of these cells in the MBH were induced with Cre (data not shown). By profiling these IKK-β knockout mice and matched controls, both of which were generated at a middleold age, IKK-β ablation in microglia prevented the increase of microglial cells over aging (FIG. 3d). Moreover, IKK-β ablation prevented aging from inducing TNF-α expression not only in microglia but also in neighbouring cells. Such aging-related hypothalamic microglianeuron crosstalk via IKK-β and NF-κB led to the prediction that microglia-specific IKK-β ablation might slow down aging. To test this prediction, IKK-β knockout mice generated at middleold age were maintained until old age, and their aging manifestations were assessed. After technical evaluation, these mice were tested using the Morris water maze; the data showed that microglia-specific IKK-β ablation reduced aging-related cognitive decline (FIG. 3e-g). Furthermore, IKK-β ablation resulted in improvements in aging-related muscle weakness (FIG. 3h) and tail collagen cross-linking (FIG. 3i). Altogether, hypothalamic microglia can act via IKK-β and NF-κB to contribute to the role of the hypothalamus in aging development.

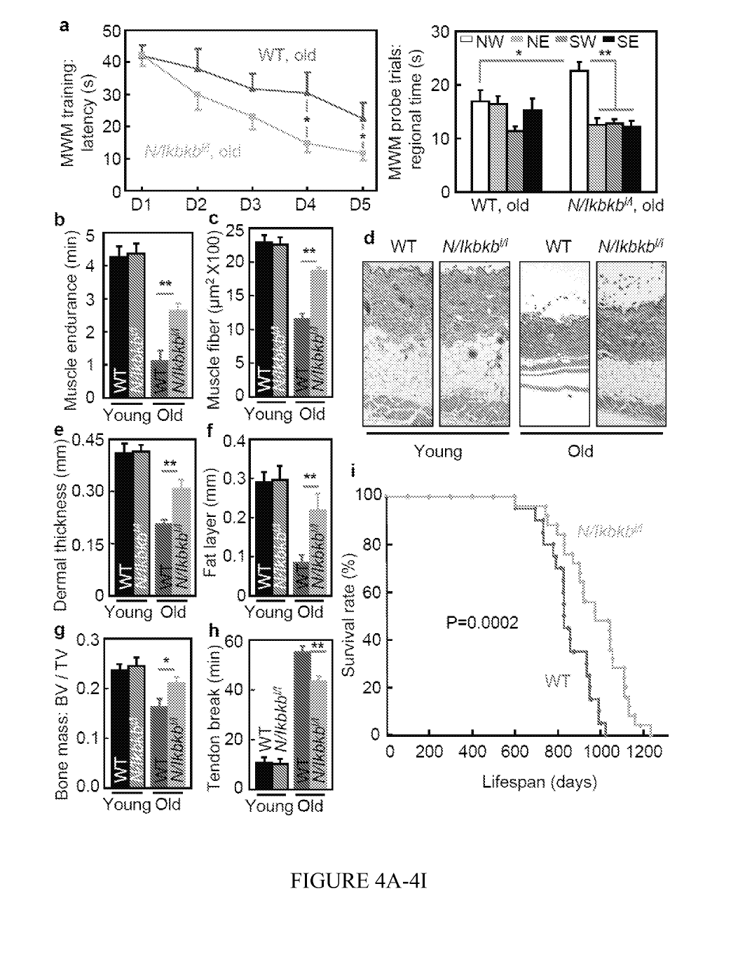

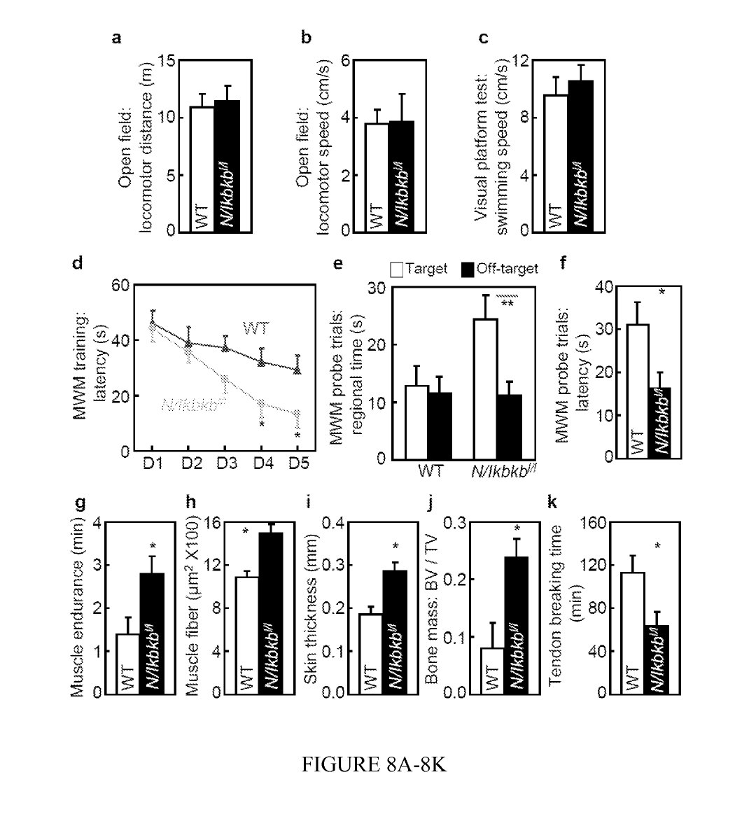

Genetic longevity by suppressing brain IKK-β. A genetic model of brain-specific IKK-β knockout mice, N/Ikbkb^{lox/lox} mice was generated by breeding nestin-Cre with Ikbkb^{lox/lox} mice as described previously¹³. Compared to wild-type littermates with −matched Ikbkb^{lox/lox} background, these knockout mice were developmentally indistinguishable in terms of brain size and gross morphology (data not shown). Ikbkb^{lox/lox} mice were compared to additional types of control; all these mice were similar across a spectrum of aging-related physiological and histological changes (data not shown). In this context, aging-related physiology and pathology were profiled in N/Ikbkb^{lox/lox} mice and littermate wild types. At an old age, after technical assessment (data not shown), mice were subjected to the Morris water maze; N/Ikbkb^{lox/lox} mice outperformed wild types (FIG. 4a). This cognitive improvement was specific to aging, because young N/Ikbkb^{lox/lox} mice and wild types performed similarly (data not shown). Thus, although NF-κB seems to have a role in the development of hippocampal synaptic plasticity³¹⁻³³, the net effect from suppressing brain IKK-β and NF-κB under the aging model is cognitively beneficial. Using a grip test, compared to wild type, N/Ikbkb^{lox/lox} mice had a reduced extent of aging-related muscle weakness (FIG. 4b). Also, as shown in FIG. 4c-h, N/Ikbkb^{lox/lox} mice were protected against aging-induced muscle and skin atrophy, bone loss and collagen cross-linking. In addition to males, female N/Ikbkb^{lox/lox} mice were studied, and the findings were consistent (FIG. 8). Lifespan analysis was conducted by following a cohort of male N/Ikbkb^{lox/lox} mice and wild-type littermates. As shown in FIG. 4i, wild-type mice had a typical pattern of median and maximal lifespan; by contrast, N/Ikbkb^{lox/lox} mice showed a pronounced phenotype of longevity, with median lifespan 23% longer (P=0.0002) and maximal lifespan 20% longer (P<0.05) than wild types. The longevity phenotype of this genetic model could be a result of IKK-β inhibition jointly in neurons and glia, as nestin-Cre is known to target neural stem/progenitor cells and derived neurons and glia. To summarize, longevity in this genetic model considerably recapitulates aging retardation from hypothalamic IKK-β and NF-κB inhibition, and technologically, aging retardation can be achieved via IKK-3 and NF-κB inhibition across the brain without evident side effects or compromised efficacy.

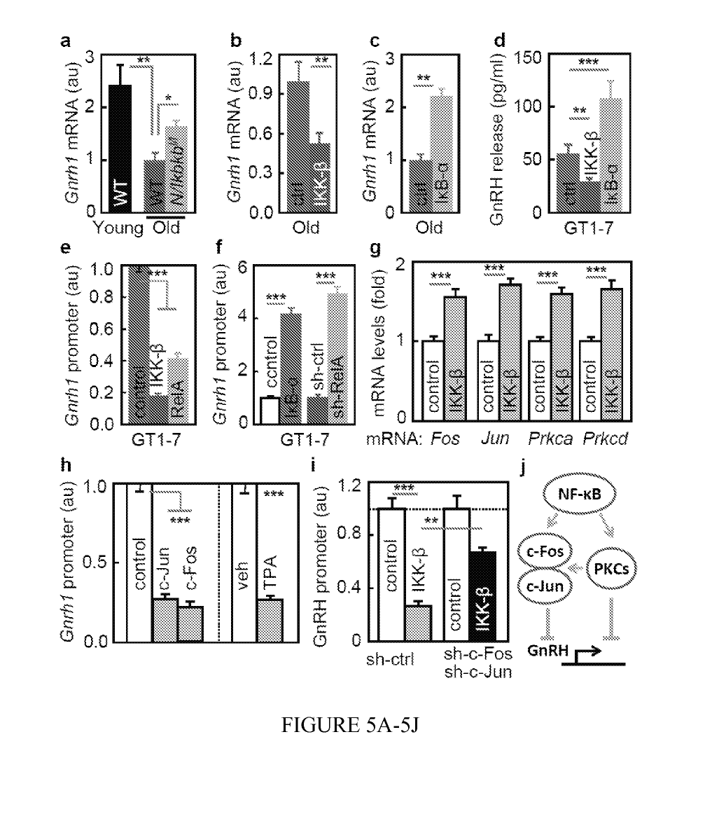

Aging-related NF-κB-induced GnRH decline. To better depict the hypothalamic control of aging, neuroendocrine pathways of the hypothalamus were investigated. IKK-β and NF-κB were found to negatively regulated GnRH. The classical action of GnRH is to regulate sex hormones and reproduction, but whether GnRH is important for whole-body aging had yet to be determined. Aging is associated with reduced hypothalamic Gnrh1 mRNA, and this change was reversed by IKK-β and NF-κB inhibition but enhanced by their activation (FIG. 5a-c). Using GT1-7 cells, a cell line of GnRH neurons, GnRH release from these cells was found to decrease after IKK-β and NF-κB activation, but increased after IKK-β and NF-κB inhibition (FIG. 5d). To study whether NF-κB might inhibit the Gnrh1 gene, Gnrh1 promoter-driven luciferase was introduced into GT1-7 cells, and IKK-β and NF-κB were simultaneously activated or inhibited in these cells. Results showed that Gnrh1 promoter activity was reduced 50% after IKK-β and NF-κB activation, but was increased 4-5-fold by IKK-β and NF-κB inhibition (FIGS. 5e, f). Moreover, IKK-β and NF-κB activation increased Fos (also known as c-fos), Jun (c-jun), Prkca (Pkca) and Prkcd (PKCδ) mRNA levels (FIG. 5g). This finding was relevant because c-Fos and c-Jun overexpression and protein kinase C (PKC) activation were both able to inhibit the Gnrh1 promoter (FIG. 5h). Furthermore, IKK-β and NF-κB inhibition of the Gnrh1 promoter was attenuated by blocking c-Fos and c-Jun (FIG. 5i) or by suppressing the PKC pathway (data not shown). Altogether, the c-Fos, c-Jun and PKC pathways can work together to mediate the inhibitory effect of IKK-β and NF-κB on GnRH (FIG. 5j), and in conjunction with relevant literature³⁴, transcriptional integration of NF-κB and c-Jun seems to account for downregulation of GnRH in the hypothalamus.

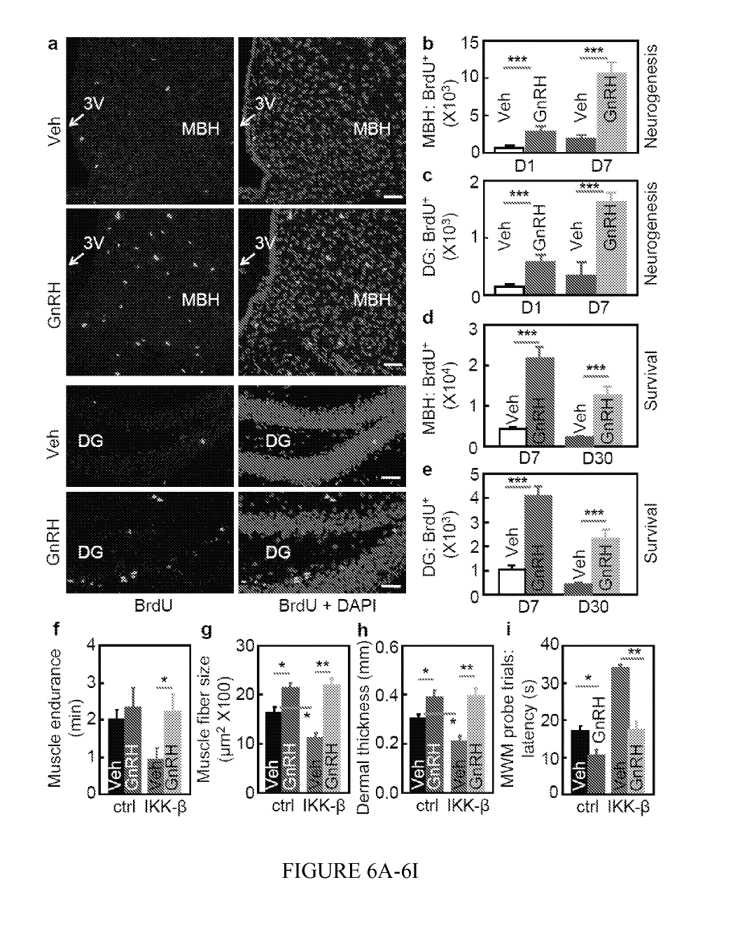

GnRH treatment prevents aging-impaired neurogenesis. On the basis of the known role of GnRH in regulating sex hormones, GnRH changes in the mouse models might correlate with changes in sex hormones; this prediction was proved (data not shown). However, a sex hormone may not be a primary mediator for aging phenotypes in the models, because hypothalamic IKK-β and NF-κB are important for aging in both sexes. Based on this, it was proposed that GnRH works as a primary mediator independently of a specific sex hormone. To explore whether GnRH exerts intra-brain actions to affect aging, GnRH was delivered into the hypothalamic third-ventricle of old mice, and aging-related changes in brain cell biology were examined A notable observation was that GnRH promoted adult neurogenesis despite aging. Using BrdU tracking following a single BrdU injection to report neurogenesis¹², aging was found to be characterized by diminished neurogenesis, particularly in the hypothalamus and the hippocampus; however, this defect was substantially reversed by GnRH treatment (FIG. 6a-c). Thirty-day BrdU tracking (with 7 days of daily BrdU injections) also confirmed that BrdU-labelled cells in GnRH-treated mice significantly survived (FIGS. 6d, e). Of note, these effects were seen in not only the hypothalamus but also the hippocampus and other brain regions (data not shown), reflecting the fact that GnRH travels within the brain to promote neurogenesis. Therefore, given the leadership role of the brain in controlling whole-body physiology, the brain-wide neurogenesis induced by hypothalamic GnRH may provide an explanation about how the hypothalamus, a very small structure in the brain, could control systemic aging.

GnRH therapy decelerates aging development. Finally, to study whether GnRH could affect aging, old MBH-IKK-β mice and MBH-ctrl mice described in FIG. 2 were subjected to daily GnRH therapy for a prolonged period, and their aging physiology and histology were then examined. In order to test whether GnRH could act peripherally to affect aging, mice were treated with GnRH via peripheral injections. Notably, GnRH treatment reduced the magnitude of aging histology in control mice and abrogated the pro-aging phenotype in MBH-IKK-βmice (FIG. 6f-h). Interestingly, despite the peripheral administration, GnRH led to an amelioration of aging-related cognitive decline (FIG. 6i). Thus, a prolonged increase of systemic GnRH can cumulatively yield actions on the brain. GnRH-responsive brain regions outside of the blood-brain barrier, such as the median eminence, subfornical organ and area postrema, can have access to peripheral-delivered GnRH. These effects of GnRH were not specific to gender, as similar outcomes were shown in males (FIG. 6f-i,) and females (data not shown). For comparison, MBH-IκB-α mice were treated with GnRH. GnRH did not further enhance the anti-aging phenotype in MBH-IκB-α mice (data not shown), suggesting that NF-κB inhibition and GnRH action may work in the same pathway to counteract aging. This body of data leads to the conclusion that the hypothalamus can integrate NF-κB-directed immunity and GnRH-driven neuroendocrine system to program aging development.

### Discussion

In this work, it was conceived that the hypothalamus, which is known to have fundamental roles in growth, development, reproduction and metabolism, is also responsible for systemic aging and thus lifespan control. Notably, through activating or inhibiting immune pathway IKK-β and NF-κB in the hypothalamus of mice, the aging process was accelerated or decelerated leading to shortened or increased lifespan. Thus, in line with literature that appreciated the effects of the nervous system on lifespan⁷⁻¹¹, the present findings provide a proof of principle to the hypothesis that aging is a life event that is programmed by the hypothalamus. Indeed, brain change is an early aging manifestation⁴, and it was reasoned that some hypothalamic alterations may act to motivate aging of the rest of the body, and that this outreaching role of the hypothalamus aligns with the fact that it is the neuroendocrine ‘headquarters’ in the body. Along this line, these studies further revealed a direct link between IKK-β and NF-κB activation and GnRH decline, and also importantly, discovered that GnRH induces adult neurogenesis broadly in the brain and that GnRH therapy can greatly amend aging disorders. Thus, while the inhibition of GnRH by NF-κB may lead to the end of reproductive length—which seems necessary for species' quality—it initiates systemic aging at the same time.

To summarize, the present study using several mouse models demonstrates that the hypothalamus is important for systemic aging and lifespan control. This hypothalamic role is significantly mediated by IKK-β- and NF-κB-directed hypothalamic innate immunity involving microglia-neuron crosstalk. The underlying basis includes integration between immunity and neuroendocrine system of the hypothalamus, and immune inhibition and GnRH restoration in the hypothalamus or the brain represent two potential strategies for combating aging-related health problems.

### REFERENCES

1. Miller, R. A. Genes against aging. J. Gerontol. A Biol. Sci. Med. Sci. 67A, 495-502 (2012).
2. Mattson, M. P. Pathways towards and away from Alzheimer's disease. Nature 430, 631-639 (2004).
3. Masoro, E. J. Overview of caloric restriction and aging. Mech. Aging Dev. 126, 913-922 (2005).
4. Finch, C. E. Neurons, glia, and plasticity in normal brain aging. Adv. Gerontol. 10, 35-39 (2002).
5. Zitnik, G. & Martin, G. M. Age-related decline in neurogenesis: old cells or old environment? J. Neurosci. Res. 70, 258-263 (2002).
6. Martin, G. M. Epigenetic gambling and epigenetic drift as an antagonistic pleiotropic mechanism of aging. Aging Cell 8, 761-764 (2009).
7. Bishop, N. A. & Guarente, L. Two neurons mediate diet-restriction-induced longevity in C. elegans. Nature 447, 545-549 (2007).
8. Fridell, Y. W., Sanchez-Blanco, A., Silvia, B. A. & Helfand, S. L. Targeted expression of the human uncoupling protein 2 (hUCP2) to adult neurons extends life span in the fly. Cell Metab. 1, 145-152 (2005).
9. Alcedo, J. & Kenyon, C. Regulation of C. elegans longevity by specific gustatory and olfactory neurons. Neuron 41, 45-55 (2004).
10. Wolkow, C. A., Kimura, K. D., Lee, M. S. & Ruvkun, G. Regulation of C. elegans life-span by insulin-like signaling in the nervous system. Science 290, 147-150 (2000).
11. Taguchi, A., Wartschow, L. M. & White, M. F. Brain IRS2 signaling coordinates life span and nutrient homeostasis. Science 317, 369-372 (2007).
12. Li, J., Tang, Y. & Cai, D. IKKβ/NF-κB disrupts adult hypothalamic neural stem cells to mediate a neurodegenerative mechanism of dietary obesity and pre-diabetes. Nature Cell Biol. 14, 999-1012 (2012).
13. Zhang, X. et al. Hypothalamic IKKβ/NF-κB and ER stress link overnutrition to energy imbalance and obesity. Cell 135, 61-73 (2008).
14. Purkayastha, S. et al. Neural dysregulation of peripheral insulin action and blood pressure by brain endoplasmic reticulum stress. Proc. Natl Acad. Sci. USA 108, 2939-2944 (2011).
15. Purkayastha, S., Zhang, G. & Cai, D. Uncoupling the mechanisms of obesity and hypertension by targeting hypothalamic IKK-β and NF-κB. Nature Med. 17, 883-887 (2011).
16. Okun, E., Griffioen, K. J. & Mattson, M. P. Toll-like receptor signaling in neural plasticity and disease. Trends Neurosci. 34, 269-281 (2011).
17. Glass, C. K., Saijo, K., Winner, B., Marchetto, M. C. & Gage, F. H. Mechanisms underlying inflammation in neurodegeneration. Cell 140, 918-934 (2010).
18. Saijo, K. et al. A Nurrl/CoREST pathway in microglia and astrocytes protects dopaminergic neurons from inflammation-induced death. Cell 137, 47-59 (2009).
19. Saijo, K., Collier, J. G., Li, A. C., Katzenellenbogen, J. A. & Glass, C. K. An ADIOL-ERβ-CtBP transrepression pathway negatively regulates microglia-mediated inflammation. Cell 145, 584-595 (2011).
20. Saijo, K. & Glass, C. K. Microglial cell origin and phenotypes in health and disease. Nature Rev. Immunol. 11, 775-787 (2011).
21. Lucin, K. M. & Wyss-Coray, T Immune activation in brain aging and neurodegeneration: too much or too little? Neuron 64, 110-122 (2009).
22. Villeda, S. & Wyss-Coray, T. Microglia-a wrench in the running wheel? Neuron 59, 527-529 (2008).
23. Villeda, S. A. et al. The aging systemic milieu negatively regulates neurogenesis and cognitive function. Nature 477, 90-94 (2011).
24. Yoshiyama, Y. et al. Synapse loss and microglial activation precede tangles in a P301S tauopathy mouse model. Neuron 53, 337-351 (2007).
25. Adler, A. S. et al. Motif module map reveals enforcement of aging by continual NF-κB activity. Genes Dev. 21, 3244-3257 (2007).
26. Peng, B. et al. Defective feedback regulation of NF-κB underlies Sjogren's syndrome in mice with mutated κB enhancers of the IκBα promoter. Proc. Natl Acad. Sci. USA 107, 15193-15198 (2010).
27. Harrison, D. E. et al. Rapamycin fed late in life extends lifespan in genetically heterogeneous mice. Nature 460, 392-395 (2009).
28. Barger, S. W. et al. Tumor necrosis factors α and β protect neurons against amyloid β-peptide toxicity: evidence for involvement of a κ B-binding factor and attenuation of peroxide and Ca2+ accumulation. Proc. Natl Acad. Sci. USA 92, 9328-9332 (1995).
29. Bruce, A. J. et al. Altered neuronal and microglial responses to excitotoxic and ischemic brain injury in mice lacking TNF receptors. Nature Med. 2, 788-794 (1996).
30. Taoufik, E. et al. Transmembrane tumour necrosis factor is neuroprotective and regulates experimental autoimmune encephalomyelitis via neuronal nuclear factor-κB. Brain 134, 2722-2735 (2011).
31. Kaltschmidt, B. et al. NF-κB regulates spatial memory formation and synaptic plasticity through protein kinase A/CREB signaling. Mol. Cell. Biol. 26, 2936-2946 (2006).
32. Meffert, M. K., Chang, J. M., Wiltgen, B. J., Fanselow, M. S. & Baltimore, D. NF-κB functions in synaptic signaling and behavior. Nature Neurosci. 6, 1072-1078 (2003).
33. O'Mahony, A. et al. NF-κB/Rel regulates inhibitory and excitatory neuronal function and synaptic plasticity. Mol. Cell. Biol. 26, 7283-7298 (2006).
34. Huang, W., Ghisletti, S., Perissi, V., Rosenfeld, M. G. & Glass, C. K. Transcriptional integration of TLR2 and TLR4 signaling at the NCoR derepression checkpoint. Mol. Cell 35, 48-57 (2009).
35. Meng, Q. & Cai, D. Defective hypothalamic autophagy directs the central pathogenesis of obesity via the IκB kinase β (IKKβ)/NF-κB pathway. J. Biol. Chem. 286, 32324-32332(2011).
36. Banks, W. A. et al. Effects of a growth hormone-releasing hormone antagonist on telomerase activity, oxidative stress, longevity, and aging in mice. Proc. Natl Acad. Sci. USA 107, 22272-22277 (2010).
37. Tillerson, J. L. & Miller, G. W. Grid performance test to measure behavioral impairment in the MPTP-treated-mouse model of parkinsonism. J. Neurosci. Methods 123, 189-200 (2003).
38. Mueller, J. M. & Pahl, H. L. Assaying NF-κB and AP-1 DNA-binding and transcriptional activity. Methods Mol. Biol. 99, 205-216 (2000).
39. Flurkey, K., Papaconstantinou, J., Miller, R. A. & Harrison, D. E. Lifespan extension and delayed immune and collagen aging in mutant mice with defects in growth hormone production. Proc. Natl Acad. Sci. USA 98, 6736-6741 (2001).
40. Ramanadham, S. et al. Age-related changes in bone morphology are accelerated in group VIA phospholipase A2 (iPLA2β)-null mice. Am. J. Pathol. 172, 868-881 (2008).
41. Meylan, E. et al. Requirement for NF-κB signalling in a mouse model of lung adenocarcinoma. Nature 462, 104-107 (2009).
42. Wang, C., Li, Q., Redden, D. T., Weindruch, R. & Allison, D. B. Statistical methods for testing effects on “maximum lifespan”. Mech. Aging Dev. 125,629-632 (2004).
43. Demetrius, L. Aging in mouse and human systems. A comparative study. Ann. N.Y. Acad. Sci. 1067: 66-82 (2006).
44. Andreollo, N. et al., Rat's age versus human's age: what is the relationship? ABCD Arg Bras Cir Dig 25(1):49-51 (2012).

1. A method of treating an aging effect in a subject comprising administering to the subject gonadotropin-releasing hormone (GnRH) or an analog thereof in an amount that is effective to treat an aging effect in a subject.

2. The method of claim 1, wherein GnRH or an analog thereof is administered to the central nervous system or to the cerebral spinal fluid of the subject.

3. The method of claim 1, wherein GNRH or an analog thereof is administered systemically to the subject.

4. A method of treating an aging effect in a subject comprising promoting neurogenesis in the brain of the subject in an amount that is effective to treat an aging effect in a subject.

5. The method of claim 4, wherein neurogenesis is promoted in the hypothalamus of the subject.

6. The method of claim 4, wherein neurogenesis is promoted in the hippocampus and/or supra-ventricular zone of the brain of the subject.

7. A method of screening for a candidate agent that treats an aging effect in a subject comprising determining whether or not the agent inhibits IκB kinase-β (IKK-β) and/or nuclear factor κB (NF-κB) in the brain, wherein an agent that inhibits IKK-β and/or NF-κB is a candidate agent for treating an aging effect in a subject.

8. The method of claim 8, wherein the agent inhibits IKK-β and/or NF-κB in the hypothalamus.

9. The method of claim 8, wherein the agent inhibits IKK-β and/or NF-κB in the hippocampus and/or supra-ventricular zone of the brain.

10. The method of claim 7, wherein the screening is carried out using a middle aged mouse or an elderly mouse.

11. The method of claim 1, wherein the aging effect comprises one or more of cognitive decline, muscle loss and/or muscle weakness, and loss of bone mass.

12. The method of claim 1, wherein the subject is a middle-aged subject.

13. The method of claim 12, wherein the middle-aged subject is a human subject at least 30 years of age.

14. The method of claim 1, wherein the subject is an elderly subject.

15. The method of claim 14, wherein the elderly subject is a human subject at least 60 years of age.
